(19) United States
(12) Patent Application Publication
(10) Pub. No.: US 2026/0314987 A1
(43) Pub. Date: Oct. 15, 2026

(54) SYSTEM AND METHOD FOR NONINVASIVE DETECTION AND CHARACTERIZATION OF CORTICAL SPREADING DEPRESSION WAVES

(71) Applicants: CARNEGIE MELLON UNIVERSITY, Pittsburgh, PA (US); University of Pittsburgh - Of the Commonwealth System of Higher Education, Pittsburgh, PA (US)

(72) Inventors: Alireza Chaman Zar, Pittsburgh, PA (US); Pulkit Grover, Pittsburgh, PA (US); Han Yi Wang, Pittsburgh, PA (US); Krushna Ashishbhai Thakkar, Pittsburgh, PA (US); Swaroop Thammineni, Pittsburgh, PA (US)

(73) Assignees: CARNEGIE MELLON UNIVERSITY, Pittsburgh, PA (US); University of Pittsburgh - Of the Commonwealth System of Higher Education, Pittsburgh, PA (US)

(21) Appl. No.: 19/703,414

(22) Filed: Jun. 10, 2026

Related U.S. Application Data
(63) Continuation-in-part of application No. 18/011,351, filed on Apr. 27, 2022, now Pat. No. 12,661,054.
(60) Provisional application No. 63/183,156, filed on May 3, 2021.

(51) Int. Cl. A61B 5/372; A61B 5/00

(52) U.S. Cl. CPC A61B 5/372; A61B 5/7267

(57) ABSTRACT

Disclosed herein is an end-to-end spatial-temporal graph attention network for the noninvasive detection and characterization of CSDs, enabling the quantitative estimation of propagation speed and cortical origin. A speed estimation task is formulated as a bounded regression problem with Huber loss, enabling quantitative and physically meaningful predictions that preserve the metric structure of propagation dynamics. An origin estimation is based on a geometry-aware framework to regress angular coordinates on a spherical cortical manifold optimized via geodesic loss.

### Related Applications

This application is a continuation-in-part application filed under 35 U.S.C. § 120 of U.S. patent application Ser. No. 18/011,351, filed Dec. 19, 2022, which is a filing under 35 U.S.C. § 371 of PCT/US2022/026524, filed Apr. 27, 2022, which claims the benefit of U.S. Provisional Patent Application No. 63/183,156, filed May 3, 2021. The contents of these applications are incorporated herein by reference in their entireties.

### BACKGROUND OF THE INVENTION

Cortical spreading depolarizations (CSDs) are propagating waves of neural suppression and a potentially treatable mechanism of secondary brain damage following traumatic brain injury and stroke. Detection and characterization of these waves can predict progressive brain damage, tailor treatments, and improve outcomes.

Noninvasive CSD monitoring using scalp electroencephalography (EEG) provides global coverage of the whole brain and enables continuous monitoring at Intensive Care Unit (ICU). The parent application, established the feasibility of noninvasive CSD detection in brain injury patients. However, noninvasive estimation of clinically relevant CSD parameters such as width of suppression, propagation speed, and cortical origin remains elusive. This is in part due to the challenges specific to the physics of the problem, for example, noisy and spatially low-passed filtered scalp EEG measurements.

### SUMMARY OF THE INVENTION

Disclosed herein as a first embodiment of the invention, is a non-invasive deep learning approach for tracking cortical spreading depressions (CSDs) in scalp electroencephalography (EEG) signals. The method, which is referred to herein as a CSD spatially aware convolutional network or CSD-SpArC, combines a convolutional neural network, which extracts temporal features from the EEG signal of each EEG electrode, with a graph neural network, which exploits the spatial structure of EEG signals on the scalp.

Using EEG, this combination of networks is able to detect even the narrowest CSDs (informed by widths observed in the real world), with less than a 1.3% “post-stitching” false alarm rate. The network is further used to track CSD wave propagation by detecting when the recording at each electrode is affected substantially by the propagating wave, quantifying its spatio-temporal tracking accuracy. The network is scalable to different densities of EEG electrodes and is generalizable to different head models.

The objective of the invention is to detect when the CSD occurs and to track which EEG electrodes are most affected by CSDs as they propagate.

Disclosed herein as a second embodiment of the invention, is an end-to-end spatial-temporal graph attention (STGAT) network for noninvasive detection and characterization of CSDs, enabling the quantitative estimation of propagation speed and cortical origin. The speed estimation task is formulated as a bounded regression problem with Huber loss, enabling quantitative and physically meaningful predictions that preserve the metric structure of propagation dynamics. The origin estimation is based on a geometry-aware framework to regress angular coordinates on a spherical cortical manifold optimized via geodesic loss.

The disclosed invention achieves a localization error of under 10 mm for cortical origin and a normalized error below 14% for propagation speed using a clinical 20-electrode ICU montage, improving to 5 mm and 6% using a high-density 256-electrode system. Thus, end-to-end spatio-temporal graph learning provides a scalable and robust framework for noninvasive estimation of clinically relevant CSD parameters, providing a physics-motivated data-driven framework for noninvasive CSD characterization.

### BRIEF DESCRIPTION OF THE DRAWINGS

FIG. 1 is a block diagram of the first embodiment of the invention showing one exemplary architecture of a model for detecting CSD.

FIG. 2 is a block diagram of a second embodiment of the invention showing a unified spatio-temporal graph attention network for noninvasive CSD parameter estimation.

FIG. 3 is a block diagram of the signal processing pipeline for EEG-based CSD modeling.

FIG. 4 is a listing of an exemplary algorithm for performing speed estimation.

FIG. 5 is a listing of an exemplary algorithm for performing origin estimation.

### DETAILED DESCRIPTION

Disclosed herein as the first embodiment of the invention is an automated, generalizable model for tracking CSDs using EEG. Two notions of generalizability are defined. First, generalizability to different patients (head models), which means that the trained model can be applied to new patients with little or no retraining of the model. Second, scalability to different densities of electrodes, which means that the model, when trained on a specific electrode density, can be applied, with little or no retraining, to other electrode densities to track CSDs.

The model combines convolutional neural networks (CNNs) and graph neural networks (GNNs). The CNNs have local connections and shared weights that extract localized multi-scale features and construct highly-expressive representations based on regular grids of data (e.g., images, texts, and time series). The GNNs are a generalization of CNNs to irregular grids (i.e., graph structures such as 3D meshes). EEG recordings are temporal on a regular grid (i.e., a 1D time series at each electrode), where a CNN is used to extract temporal features for each electrode, and spatial on an irregular grid (i.e., a 3D electrode mesh), where a GNN is utilized to extract spatial information.

The spatio-temporal nature of the information of CSD waves in EEG signals using a deep learning framework is exploited by the invention. The deep learning framework comprises: (1) a CNN to extract temporal features from the preprocessed signals of each electrode; (2) a GNN to aggregate the temporal features and to exploit the spatial structure of electrode locations on the scalp to extract spatial information; and (3) a classifier, for example, a multilayer perceptron (MLP), to classify the presence or absence of CSD wavefronts at each electrode location in small non-overlapping time intervals.

The scalability of the disclosed invention, which is referred to herein as CSD-SpArC (CSD spatially aware convolutional network), has been proven using simulations on a wide range of EEG electrode densities, from a low-density grid of 20-electrode standard EEG caps, to higher densities with up to 256 electrodes at 10-5 standard locations. This makes it possible to exploit the large amount of low-density EEG data from continuous recordings at intensive care units to train the CSD-SpArC for higher densities of EEGs, for which there is not much CSD data available. Additionally, the generalizability has been proven with respect to a plurality of different head models. This provides a system that can provide rapid detection of CSDs for patients as they arrive at hospitals.

The dataset generation process, the data labeling at each electrode, the EEG preprocessing steps and details of the CSD-SpArC model will now be explained.

### Dataset Generation

EEG signals of CSD waves can be simulated using the following process: First, 3D head models of 4 healthy individuals are preprocessed and extracted from an open-source magnetic resonance imaging (MRI) dataset. Second, homogeneous annulus shaped CSD waves are simulated on a real brain model, with the origin chosen randomly on the conical surface, while excluding the bottom of the brain where there is no EEG electrode coverage. Next, spontaneous brain activity is generated using some simplifying assumptions, using a normal random process, and brain activity is suppressed at the locations and time points where CSD wavefronts are to be located. The amplitude of the suppressed signal at the simulated CSD wave is ~25% of the simulated normal brain signals. This amplitude reduction is chosen based on the average of the reported range of CSD depressions. Lastly, leadfield matrices (A) based on the extracted head models are estimated and applied to the simulated brain signals to obtain scalp EEG signals (i.e., readings).

### Binary Labeling of Data at Each Electrode Across Time for Training

The model is trained using binary labels that denote whether the signal of an electrode at a specific time point is substantially affected by CSD waves. To extract these binary labels, the brain sources corresponding to the 30% largest elements in the i^{th} row of the leadfield matrix A are examined, and, if at least at one of these sources at time t indicates a CSD depression, the label ‘T’ is assigned to the signal of the i^{th}electrode at time t.

### CSD Spatially Aware Convolutional Network (CSD-SpArC)

EEG Preprocessing—Preprocesing of the simulated EEG signals is performed to improve the signal-to-noise-ratio (SNR) of CSD depressions in the simulated EEG signals. The preprocessing techniques include Laplacian spatial filtering (for high density EEG caps with 128 and 256 electrodes), average power calculation, envelope extraction (e.g., using a sliding time window of 40 s), and cross-correlation (e.g., with 2-minute negative pulse) for each signal. In addition, the distribution of training, validation, and test datasets are normalized separately by mean subtraction and standard deviation division. The preprocessed signal is then downsampled (e.g., with a temporal step size of 6 s) before being fed into the network. This downsampling reduces the computational complexity of the network, while maintaining the required temporal information of the preprocessed signal to track CSD waves. The preprocessed signal, after the envelope extraction and cross-correlation, is concentrated in a very low frequency band. In some cases, the temporal width of depression is on the order of minutes.

Training Algorithm—A deep learning framework is used, which includes, in some embodiments, a CNN architecture called Multi Scale 1D ResNet, a GNN architecture called a graph attention network and a multilayer perceptron classifier.

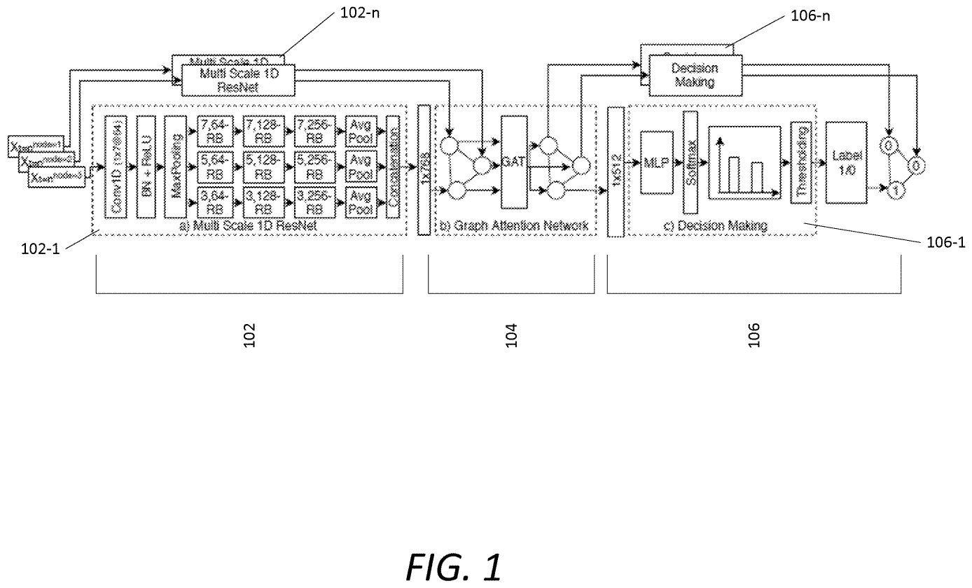

One embodiment of the model of the invention is shown in block diagram form in FIG. 1. The first part of the network comprises multiple CNNs 102-1 . . . 102-n each of which performs temporal feature extraction on preprocessed scalp EEG signals from a single EEG electrode. Each CNN 102 consists of a 1D convolution layer, an activation function which may be, for example, a ReLU function, a pooling layer and multiple residual blocks. In some embodiments, each CNN 102 may comprise a Multi-Scale 1D ResNet. The 1D convolutional filters may be of various sizes and residual connections, which are known to improve time series classification. In FIG. 1, “m, n-RB” denotes a residual block of n filters, each of length m.

CSD detection at time t is performed, in one embodiment, for non-overlapping 5-min windows of the preprocessed signals from EEG electrodes, where t is the midpoint of the window. The binary labels of the midpoints are used as the CSD ground truth for the corresponding time windows. The 5-min windows from all electrodes are fed as a batch into the ResNet to extract temporal features for each electrode.

The extracted temporal features are aggregated and input to graph attention network (GAT) 104 to exploit the spatial information in the aggregated signals. The GAT model 104 is constructed using learnable attention layers to aggregate the incoming feature vectors to a node, where the attention score calculations are based on a k-nearest neighbor (k-NN) graph. In one embodiment, the GAT comprises 6 attention heads, residual connections, 2-4 layers, 500-600 hidden units, and a LeakyReLU activation function. The k-NN graph is a geometric graph that thresholds a distance, for example, the Euclidian distance, between each pair of EEG electrodes on the scalp.

The classifiers 106-1 . . . 106-n for each node/electrode, in one embodiment, use a MLP, followed by a Softmax layer to generate the spatio-temporal probability map of CSD. For example, a threshold of 0.75 is used to extract the binary outputs (1 for “CSD” and 0 for “no CSD”) from the probability distribution of CSD produced by the network.

Finally, recognizing that CSDs only substantially affect a few electrodes during each time interval, the binary values at each electrode are stitched together, using, in one embodiment, a sliding time window of 10 min, with the assumption that each CSD spreads for at least 10 min. If there are at least 2 non-zero values in the time window, then a “temporal binary label” of 1 is assigned to the 10-min window. The consecutive “1′s” in the union of these temporal labels, across all electrodes, is declared as a single CSD episode.

Training Process—In one instance, CSD-SpArC was trained on 5,850simulated CSD episodes with varying length in the range of of 30-205 minutes, different speeds of propagation, in the range of 0-8 mm/min (with step size of 0.5 mm/min), and different widths of wavefronts in the range of 0.5-6.5 cm, (with step size of 0.5 cm), based on three different head models. The dataset included 10 simulated episodes for each combination of width, speed, and head model. For each of the different densities of EEG electrodes (20, 32, 64, 128, and 256 at 10-5 standard locations), a model is trained. To prevent overfitting, dropouts are used on all layers, except CNN, with P = 0.5. The optimal combination of hyperparameters is found through validation using an open-source hyperparameter optimization framework. The hyperparameters in CSD-SpArC are optimized over the following ranges: learning rate in (10⁻⁵, 10⁻³), k in k-NN graph for GAT in ([N/10], [N/2]) where N is the number of EEG electrodes, weight decay in (5×10⁻⁴, 10⁻²), hidden layers of GAT in (2,4), and hidden units in (400,700).

Implementation Details: CSD simulations and preprocessing may be implemented in Matlab. The model may be trained and tested in PyTorch, using a batch size of 40 windows with total training iterations of 10 epochs with early stopping on validation loss of patience of 2 epochs.

Detection Performance Metrics: The spatio-temporal tracking accuracy, defined for detecting CSDs at non-overlapping time intervals, is the degree of agreement between the binary ground truth labels of CSD and the binary outputs of the model, measured using Cohen's kappa statistic (k). The standard error of k(SE(k)) is also provided. The average false positive rate, where

$$FPR = \frac{FP}{FP + TN} ,$$

where FP is the number of false positives and TN is the number of true negatives) is reported. FPR is also reported based on the temporal binary labels (see the stitching process described above), which is referred to as “post-stitching” FPR herein.

### Proof of Concept

The performance of CSD-SpArC for generalizability, scalability, and a range of CSD widths was experimentally verified.

Performance on Varying Ranges of CSD Width-The width of CSD wavefronts varies in different neurological diseases and scenarios (e.g., 0.8 to 6.4 cm in TBI). The performance of CSD-SpArC was tested on different widths of CSD wavefronts using different densities of EEG electrodes. The trained model is tested on a simulated dataset with 4 different ranges of CSD widths (0.5-2, 2-3.5, 3.5-5 and 5-6.5 (cm) with 0.5 cm step size) and a speed range of 2 to 8 mm/min (with step size of 2 mm/min) for each width range, across three different head models and for five different EEG densities (20 to 256 electrodes). Table 1 below summarizes the results of CSD detection for different widths of wavefronts and different EEG densities. As expected, the tracking accuracy increases as the number of EEG electrodes increases and as the CSD waves become wider, with the best accuracy of 86.65%±0.60 for detecting the widest CSD waves (5-6.5 cm) using the highest density EEG (256 electrodes).

TABLE 1

| | | Trained On | | | | |
|---|---|---|---|---|---|---|
| k ± SE(k) | | 20 | 32 | 64 | 128 | 256 |
| CSD | 0.5-2 | 34.50% ± 21.87 | 40.52% ± 14.75 | 42.85% ± 10.23 | 47.71% ± 6.88 | 57.46% ± 4.10 |
| Width | 2-3.5 | 67.24% ± 6.35 | 72.56% ± 4.34 | 72.65% ± 3.07 | 72.73% ± 2.26 | 75.62% ± 1.46 |
| (cm) | 3.5-5 | 81.26% ± 3.00 | 82.40% ± 2.33 | 83.22% ± 1.63 | 83.25% ± 1.18 | 83.98% ± 0.81 |
| | 5-6.5 | 84.00% ± 2.26 | 85.94% ± 1.73 | 85.37% ± 1.25 | 85.86% ± 0.88 | 86.65% ± 0.60 |

Although non-invasive detection of CSD is challenging, the narrowest CSD waves (0.5-2 cm) can still be detected using CSD-SpArC using the highest density EEG (256 electrodes) with a tracking accuracy of 57.46% ±4.10 and an average FPR of less than 1.43%. CSD-SpArC also detects CSD waves as narrow as 2 cm using only 20 EEG electrodes with a tracking accuracy of 67.24% ±6.35 and an average FPR of less than 6.4%. Additionally, high-density EEG (256 electrodes) detect all CSD episodes, even the narrowest ones, with less than 1.3% “post-stitching” FPR.

Generalizability—To test the generalizability of CSD-SpArC to new head models (unseen by the network) the model was trained on three head models, on a simulated CSD dataset with different widths of wavefronts (0.5, 2, 5, and 6 cm) and two different speeds of propagation (2 and 4 mm/min). CSD-SpArC, trained in this manner, successfully detects and tracks CSD waves in the head model with greater than 75% tracking accuracy and less than 3% FPR using 20 or more electrodes. CSD-SpArC generalizes without any retraining and/or fine-tuning the parameters in the model.

Scalability—The model easily adapts to different densities and placements of EEG electrodes. This makes CSD-SpArC scalable to higher or lower densities of EEG electrodes. The scalability performance of CSD-SpArC was tested as follows: The trained model is used on a specific density of EEG electrodes (e.g., 20-electrodes), and was tested on a simulated test dataset of other densities (e.g., 32, 64, 128, and 256 electrodes). Each test set includes CSD episodes with different widths (0.5, 2, 4,and 6 cm) and speeds (1, 3, 5, 7 mm/min), for three different head models. Table 2shows the results of scalability tests, with the training EEG densities across columns, and test densities across rows. The results confirm the scalability of CSD-SpArC for any combination of train-test electrode densities with an accuracy of 76.7% ±1.1 and a FPR of less than 2.1% for a model which is trained on a low-density EEG (with only 20 electrodes) and tested on a high-density EEG (with 256 electrodes).

TABLE 2

| | | Trained On | | | | |
|---|---|---|---|---|---|---|
| k ± SE(k) | | 20 | 32 | 64 | 128 | 256 |
| Tested | 20 | 75.73% ± 3.99 | 77.01% ± 3.80 | 76.54% ± 3.87 | 75.22% ± 4.00 | 76.31% ± 3.72 |
| On | 32 | 75.33% ± 3.21 | 77.13% ± 3.03 | 77.06% ± 3.02 | 75.88% ± 3.15 | 77.01% ± 2.94 |
| | 64 | 75.69% ± 2.25 | 77.20% ± 2.15 | 77.42% ± 2.14 | 77.28% ± 2.16 | 77.88% ± 2.09 |
| | 128 | 75.08% ± 1.54 | 76.25% ± 1.54 | 76.54% ± 1.52 | 77.13% ± 1.74 | 76.91% ± 1.54 |
| | 256 | 76.69% ± 1.10 | 78.41% ± 1.05 | 78.24% ± 1.06 | 78.63% ± 1.07 | 79.69% ± 1.02 |

In the second embodiment of the invention, a spatio-temporal graph attention network is used for noninvasive estimation of CSD propagation speed and localization of cortical origin from scalp EEG. The speed estimation is formulated as a bounded scalar regression. Using this method, the accuracy of the estimation scales with electrode density. The origin localization is formulated as two-angle regression on a spherical cortical manifold. The second embodiment of the invention uses end-to-end spatio-temporal graph learning that enables noninvasive estimation of multiple physically meaningful CSD parameters from scalp EEG.

Let X_{w} ∈ [symbol] denote a window of scalp EEG with N electrodes and W temporal samples. Electrodes are modeled as nodes of a graph [symbol] =( [symbol] , Ε) constructed via a k-nearest-neighbor (k=3) rule on inter-electrode distances, with self-loops to preserve node-specific information. Given a five-minute nonoverlapping window, a unified mapping is learned:

f_{θ}=X_{w} →h→ [symbol] (1)

where h∈ [symbol] is a learned spatio-temporal representation and [symbol] is the task-specific output:

Speed Estimation: [symbol] =s∈ [symbol] (2)

Origin Estimation: [symbol] =(αₓ, α_{y})∈ [symbol] (3)

where s is the CSD propagation speed (mm/min) and (aₓ, a_{y}) parameterize the cortical origin via rotation angles. Both tasks share a common backbone extracting graph-level embedding h, followed by task-specific prediction heads. For configurations with more than 64 electrodes, surface Laplacian spatial filtering is applied.

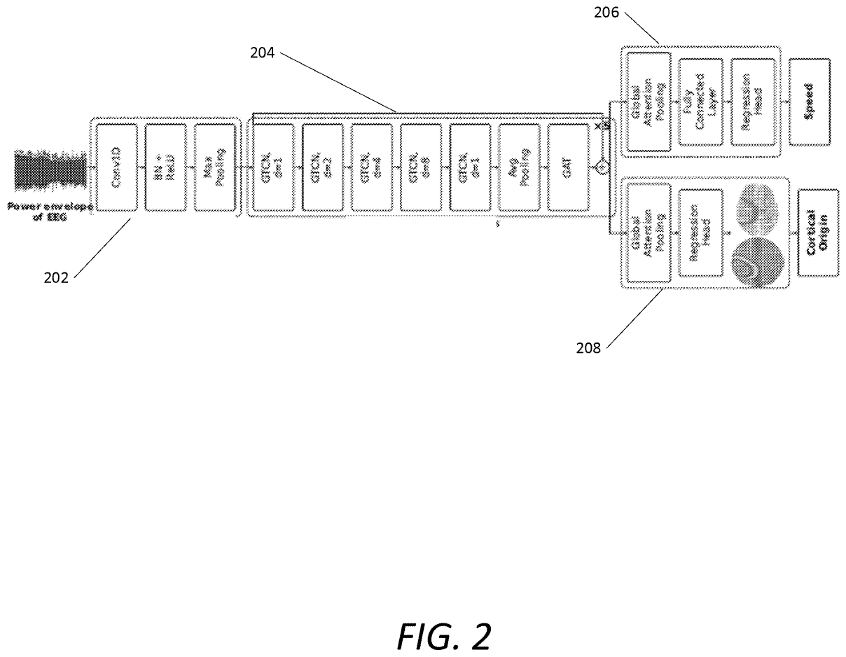

A block diagram of the inified spatio-temporal graph attention architecture for noninvasive CSD parameter estimation is shown in FIG. 2. EEG signals are processed through a temporal convolutional feature extractor 202, passed through multiple spatio-temporal blocks 204, where dilated temporal convolutions capture multi-scale propagation patterns and graph attention layers model spatial dependencies. For speed estimation, at 206, node embeddings from spatio-temporal backbone 204 are aggregated via global attention pooling to produce a graph-level representation which is mapped to a scalar speed estimate. For origin estimation, at 208, node embeddings from the same spatio-temporal backbone 204 are aggregated via global attention pooling and mapped to angular coordinates (αₓ, α_{y}) via a bounded regression head, then projected onto the spherical cortical surface, enabling accurate localization even under sparse and noisy electrode configurations.

Spatio-Temporal Graph Attention (STGAT) Backbone—A spatial-temporal graph attention network is used to model EEG dynamics. The backbone operates on graph [symbol] and alternates between temporal feature extraction and spatial aggregation.

Given input X_{w}, node-wise temporal features are computed. Information is then propagated across the electrode graph, and node embeddings are aggregated into a graph-level representation:

$$h = Pool ( {\{ {h}_{i}^{L} \}}_{i = 1}^{N} ) \qquad ( 4 )$$

where

$${h}_{i}^{L}$$

is the final representation of node i after L layers.

Temporal Layer Modelling—Node-wise temporal dependencies are modelled using dilated gated temporal convolution networks (GTCNs), which combine dilated 1D convolutions with the gated activation mechanism of WaveNet-style architectures. Let

$${h}_{i}^{ℓ} ( t )$$

denote the feature at node i, layer [symbol] , and time index t. The dilated convolution computes:

$${\tilde{h}}_{i}^{ℓ + 1} ( t ) = {\sum }_{τ = 0}^{K - 1} {W}_{τ} {h}_{i}^{ℓ} ( t - d τ ) \qquad ( 5 )$$

where K is the kernel size and d is the dilation factor. A gating mechanism then modulates information flow:

$${h}_{i}^{ℓ + 1} ( t ) = tanh {\tilde{h}}_{i}^{ℓ + 1} ( t ) ⊙ σ ( {\tilde{g}}_{i}^{ℓ + 1} ( t ) ) \qquad ( 6 )$$

where

$${\tilde{g}}_{i}^{ℓ + 1} ( t )$$

is produced by a parallel convolution with the same dilation pattern.

Spatial Layer Modelling—Spatial dependencies are captured using graph attention networks. For each edge (i, j)∈Ε, attention coefficients are computed as:

$${e}_{i , j} = LeakyReLU ( {a}^{T} [ W {h}_{i} ‖ W {h}_{j} ] ) \qquad ( 7 )$$

$${α}_{i , j} = \frac{exp {e}_{i , j}}{{\sum }_{k ∈ 𝒩 ( i )} exp ( {e}_{i , k} )} \qquad ( 8 )$$

Node representations are updated via:

$${h}_{i}^{′} = {\sum }_{j ∈ 𝒩 ( i )} {α}_{i , j} W {h}_{j} \qquad ( 9 )$$

Layer Composition—Each ST-block combines temporal and spatial operations:

[symbol]

=GAT(TCN( [symbol] )) (10)

and multiple blocks are stacked to capture long-range dependencies.

Graph-Level Readout—Finally, node embeddings are aggregated into a graph-level representation h using a permutation-invariant pooling operator:

$$h = Pool ( {\{ {h}_{i} \}}_{i = 1}^{N} ) \qquad ( 11 )$$

which serves as input to task-specific heads.

Speed Estimation (206)—In one embodiment, EEG electrodes are represented as a kNN graph (e.g., k=3), and each input window covers five minutes of EEG. After preprocessing and downsampling, each window is represented by, for example, T=290 temporal samples, corresponding to an effective temporal resolution of approximately 1.03 seconds per sample. FIG. 1 illustrates the speed estimation pipeline. EEG signals are processed through a temporal convolutional feature extractor 202, passed through multiple spatio-temporal blocks 204, where dilated temporal convolutions capture multi-scale propagation patterns and graph attention layers model spatial dependencies, and aggregated via global attention readout to produce a graph-level representation, which is mapped to a scalar speed estimate.

CSD propagation speed is a continuous physical quantity that can take any value in a physiologically bounded range (e.g., 1-8 mm/min). Although the simulated dataset contains a finite set of discrete speed values, treating speed estimation as classification over these values would ignore the ordinal and metric structure of the underlying physical quantity and would not generalize to unseen speeds. The speed estimation is therefore formulated as bounded scalar regression, which preserves the metric structure of propagation dynamics and produces predictions on a continuous scale.

Bounded Formulation—To address this mismatch, the speed estimation is formulated as a scalar regression problem. Given the graph-level representation h, a raw scalar is predicted as:

r=f_{θ}(h) (12)

and mapped to a physically valid range using a bounded transformation:

s=sₘᵢₙ+(sₘₐₓ−sₘᵢₙ)·σ(r) (13)

where σ(·)is the sigmoid function and [sₘᵢₙ, sₘₐₓ]=[0.996, 6.807] mm/min. This ensures that predictions remain within physiologically meaningful limits.

Loss Function-The model is trained using Huber loss:

$$e = \hat{s} - s , {ℒ}_{speed} ( \hat{s} , s ) = \{ \begin{matrix}\frac{1}{2} {e}^{2} , if \vert e \vert \leq δ \\ δ \vert e \vert - \frac{1}{2} {δ}^{2} otherwise\end{matrix} \qquad ( 14 )$$

with δ=5.

The Huber loss combines the sensitivity of squared error for small deviations with the robustness of absolute error for large deviations, reducing the influence of outliers while maintaining stable gradients. This is particularly important in a setting where estimation noise and variability in simulated propagation patterns can lead to occasional large residuals.

Evaluation Metric—The performance can be evaluated using normalized error (NE):

$$NE = \frac{\vert \hat{s} - s \vert}{s} \qquad ( 15 )$$

which measures relative deviation from the true propagation speed. This metric is scale-invariant and reflects the clinical relevance of proportional errors, ensuring that deviations at lower speeds are appropriately weighted relative to higher-speed regimes.

Origin Estimation on a Spherical Manifold (208)—The cortical surface is modelled as a sphere of mean radius (e.g., R=87 mm), and the CSD origin is represented as a point on the unit sphere [symbol] . The dataset parameterizes each origin using two rotation angles (αₓ, α_{y})∈[-40 °, 40 °]². A mapping is defined as:

u: [symbol] (16)

such that:

u(αₓ, α_{y})=R_{y}(α_{y})Rₓ(αₓ)p₀ (17)

where p₀=[0,0,1] and Rₓ, R_{y} are rotation operators. The learning objective is to estimate u from EEG observations.

Node embeddings from the spatio-temporal backbone 204 are aggregated via global attention pooling and mapped to angular coordinates (αₓ, α_{y}) via a bounded regression head, then projected onto the spherical cortical surface (See Eq. 16-17), enabling accurate localization even under sparse and noisy electrode configurations.

Spatio-Temporal Graph Attention (STGAT) Predictor—Given an EEG window X_{w}, the STGAT backbone produces a graph-level embedding h∈ [symbol] :

h=fθ(X_{w}, [symbol] ) (18)

A parametric regressor is then defined as:

\hat{α}=g_{θ}(h)∈ [symbol] (19)

implemented as a multi-layer perceptron with sigmoid scaling to enforce bounded outputs:

\hat{α}=αₘᵢₙ+(αₘₐₓ−αₘᵢₙ)·σ(g_{φ}(h)) (20)

This yields a prediction û=u(\hat{α}) on [symbol] .

Learning Spaces on Euclidean vs. Riemannian—Two training objectives corresponding to different geometric assumptions are considered: (A) Euclidian Angle-Space Regression; and (B) Riemannian (geodesic) Regression.

Euclidian Angle-Space Regression treats (αₓ, α_{y}) as coordinates in [symbol] :

$${ℒ}_{Euc} = {‖ \hat{α} - α ‖}_{2}^{2} \qquad ( 21 )$$

For Riemannian (geodesic) Regression, optimization occurs directly on [symbol] using the geodesic distance:

[symbol]

=R·arccos(u^{T}u) (22)

This loss corresponds to the intrinsic metric on the sphere and is differentiable via the mapping u(·). The Euclidean objective ignores curvature and may distort distances, particularly for large angular deviations. In contrast, geodesic regression respects the manifold structure, ensuring that optimization directly minimizes spatial localization error.

Loss and Metric—Training uses Huber loss (Eq. 14) with δ=0.5. The localization error is reported as the geodesic distance on the cortical sphere:

Loc.Err.=R·arccos(û^{T}u)[mm] (25)

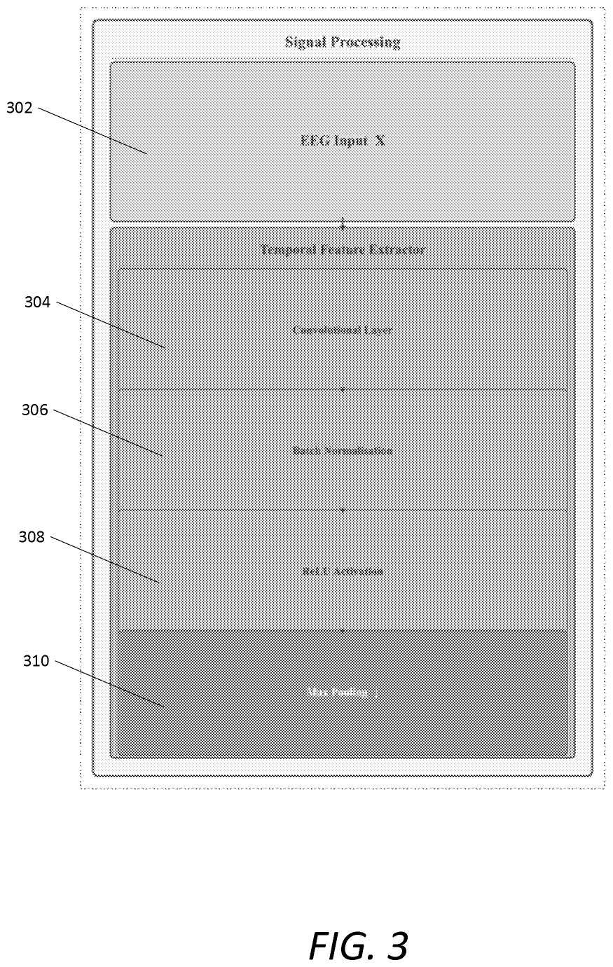

FIG. 3 is a block diagram of the signal processing pipeline for EEG-based CSD modeling. Raw EEG signals are transformed using temporal convolution, normalization, and nonlinear activation to extract robust temporal features prior to graph-based modeling.

EEG recordings 302 are modelled as spatio-temporal signals arising from propagating cortical depolarization waves. Each electrode captures a temporally evolving projection of underlying neural activity, which is spatially correlated due to wave propagation. Given raw EEG signals X∈ [symbol] , temporal features are constructed through a sequence of operations: (1) Temporal convolution 304: A 1D convolution (k=3) extracts local waveform patterns corresponding to suppression events and propagation transitions; (2) Normalization 306: Batch normalization stabilizes signal statistics across electrodes and time; (3) Nonlinearity: ReLU activation 308 introduces sparsity and enhances event-driven dynamics; and (4) Temporal max pooling 310: Downsampling reduces redundancy while preserving propagation structure.

The pipeline bridges neuroscience and machine learning by transforming raw EEG signals into representations that retain physiological propagation patterns while remaining suitable for deep spatio-temporal modeling.

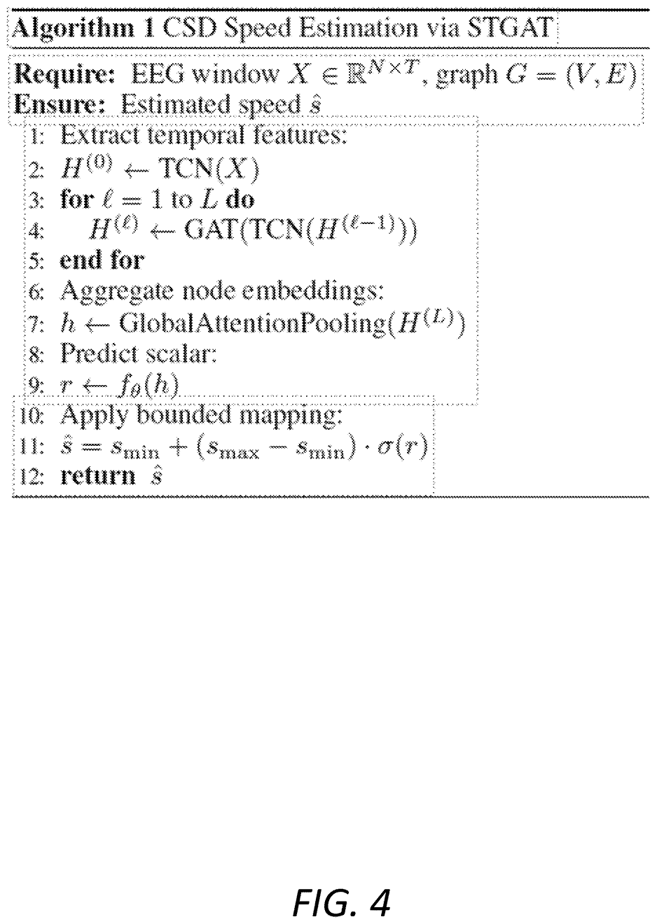

FIG. 4 is a pseudo-code listing of an exemplary algorithm for performing speed estimation. This formulation replaces classification-based approaches with bounded regression, preserving the continuous nature of propagation speed. The model learns propagation dynamics directly from full spatio-temporal signals rather than discretized bins.

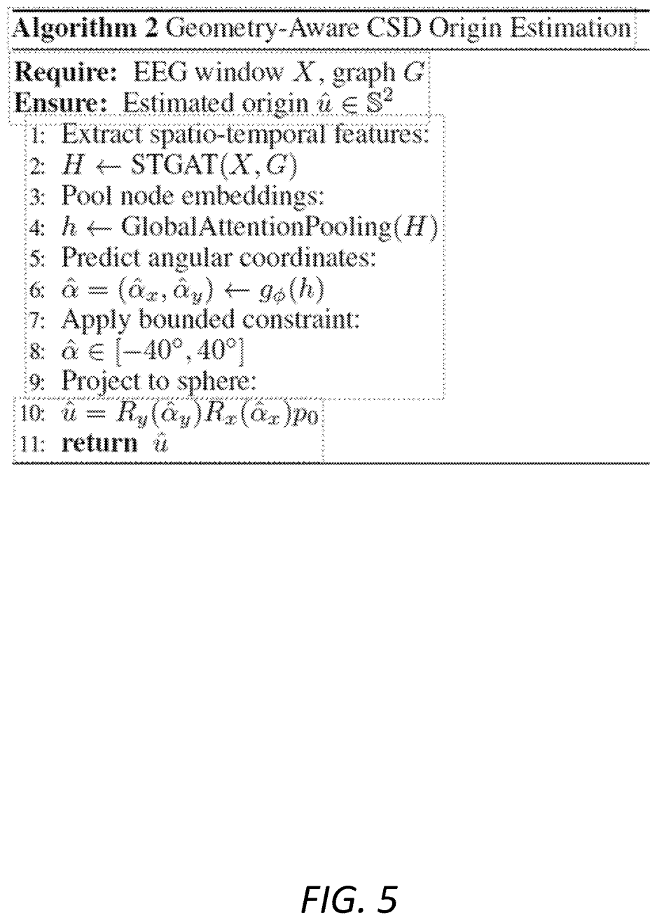

FIG. 5 is a pseudo-code listing of an exemplary algorithm for performing origin estimation. Graph-level embeddings are mapped to angular coordinates, which are projected onto a spherical cortical surface to obtain physically meaningful origin estimates. The origin estimation task is inherently geometric, as cortical locations lie on a curved manifold. The cortex is modelled as a sphere and the origin is parameterized using angular coordinates. This algorithm ensures that predictions respect the geometry of the cortical surface, unlike Euclidean regression approaches, which distort spatial relationships.

The disclosed second embodiment is the first end-to-end spatio-temporal graph learning framework for jointly estimating CSD propagation speed and cortical origin from noninvasive scalp EEG. By formulating speed as bounded regression with Huber loss and origin as geodesic regression on a spherical cortical manifold, the model achieves clinically meaningful accuracy across electrode densities from 20 to 256. End-to-end spatio-temporal graph learning can bridge the gap between noninvasive EEG measurements and clinically actionable CSD characterization, laying the algorithmic groundwork for future clinical translation.

As would be realized by one of skill in the art, the disclosed systems and methods described herein can be implemented by a system coupled to an EEG apparatus, the system comprising a processor and memory, storing software that, when executed by the processor, performs the functions comprising the method. For example, the training, testing and deployment of the model can be implemented by software executing on a processor.

As would further be realized by one of skill in the art, many variations on implementations discussed herein which fall within the scope of the invention are possible. Specifically, many variations of the architecture of the model could be used to obtain similar results. The invention is not meant to be limited to the particular exemplary model disclosed herein. Moreover, it is to be understood that the features of the various embodiments described herein were not mutually exclusive and can exist in various combinations and permutations, even if such combinations or permutations were not made express herein, without departing from the spirit and scope of the invention. Accordingly, the method and apparatus disclosed herein are not to be taken as limitations on the invention but as an illustration thereof. The scope of the invention is defined by the claims which follow.

1. A system for characterizing a cortical spreading depression wave from EEG signals comprising:
a temporal feature extractor configured to extract temporal features from an EEG power envelope;
a spatio-temporal graph attention backbone comprising one or more temporal convolutions and one or more graph attention layers configured produce node embeddings of the temporal features in a graph attention network representing a spatial configuration of electrodes of the EEG;
a speed prediction head configured to estimate speed of the cortical spreading depression wave from the node embeddings; and
an origin prediction head configured to estimate an origin of the cortical spreading depression wave from the node embeddings.

2. The system of claim 1 wherein the temporal feature extractor comprises:
a temporal convolution layer;
a batch normalization;
a nonlinear activation; and
temporal max pooling.

3. The system of claim 1 wherein the spatio-temporal graph attention backbone performs temporal modelling of node-wise temporal dependencies using a dilated gated temporal convolution network configured to combine dilated 1D convolutions with a gated activation mechanism.

4. The system of claim 1 wherein the spatio-temporal graph attention backbone performs spatial modelling of spatial dependencies using a graph attention network.

5. The system of claim 1 wherein the speed prediction head is configured to aggregate the node embeddings via global attention pooling to produce a graph-level representation which is mapped to a scalar speed estimate.

6. The system of claim 5 wherein the speed prediction head is formulated as a bounded scalar regression configured to preserve metric structure of propagation dynamics and to produce predictions on a continuous scale.

7. The system of claim 6 wherein the speed prediction head comprises:
a global attention pooling mechanism;
a fully-connected convolutional layer; and
a regression head configured to perform scalar regression.

8. The system of claim 7 wherein the convolutional layer is trained using Huber loss.

9. The system of claim 1 wherein the origin prediction head is configured to aggregate the node embeddings via global attention pooling and map the node embeddings to angular coordinates via a bounded regression head which are then projected onto a spherical cortical surface.

10. The system of claim 9 wherein the spherical cortical surface is modelled as a cortical manifold comprising a sphere of mean radius and further wherein the origin of the cortical spreading depression wave is represented as a point on the sphere.

11. The system of claim 10 wherein the origin prediction head comprises:
a global attention pooling mechanism;
a regression head configured to predict angular coordinates of the origin; and
a projection mechanism for projecting the angular coordinates onto the cortical manifold.

12. The system of claim 11 wherein the regression head is a parametric regressor implemented as a multi-layer perceptron.

13. The system of claim 12 wherein the regression head uses Euclidian angle-space regression.

14. The system of claim 12 wherein the regression head uses geodesic regression.

15. The system of claim 12 wherein the origin prediction head is trained using Huber loss.

16. The system comprising further comprising:
a processor:
an EEG apparatus comprising a plurality of EEG electrodes disposed on the head of the patient and in communication with the processor; and
software, executing on the processor, the software implementing the temporal feature extractor, the spatio-temporal graph attention backbone, the speed prediction head and the origin prediction head.

17. A method for characterizing a cortical spreading depression wave from EEG signals comprising:
extracting temporal features from an EEG power envelope;
producing node embeddings of the temporal features in a graph attention network;
estimating speed of the cortical spreading depression wave from the node embeddings; and
estimating an origin cortical spreading depression wave from the node embeddings.

18. The method of claim 17 wherein the node embeddings are produced by a gated temporal convolutional network.

19. The method of claim 17 wherein the speed estimation is produced by using a bounded scalar regression;

20. The method of claim 17 wherein the origin estimation is produced as a point of a cortical manifold modelled as a sphere.
